(19) United States
(12) Patent Application Publication
(10) Pub. No.: US 2026/0137535 A1
(43) Pub. Date: May 21, 2026

(54) APPARATUSES AND METHODS FOR EXPANDABLE BRANCHING STENT PROSTHESES

(71) Applicant: Merit Medical Systems, Inc., South Jordan, UT (US)

(72) Inventors: Michael Adams, Bluffdale, UT (US); Joanne Hislop, American Fork, UT (US)

(21) Appl. No.: 19/388,888

(22) Filed: Nov. 13, 2025

Related U.S. Application Data
(60) Provisional application No. 63/721,975, filed on Nov. 18, 2024.

(51) Int. Cl. A61F 2/954; A61F 2/958

(52) U.S. Cl. CPC A61F 2/954; A61F 2/958; A61F 2230/006

(57) ABSTRACT

A method of manipulating a deployment system is provided. The method includes, delivering a deployment system to a branching implant site. The deployment system includes an expandable stent prosthesis including a trunk portion, a first branching portion, and a second branching portion. The deployment system also includes a floss wire including a proximal portion disposed in the first branching portion, a bend disposed at a bifurcation point of the stent prosthesis, and a distal portion disposed in the second branching portion. The method further includes displacing the floss wire to seat the expandable stent prosthesis within the branching implant site, and expanding the expandable stent prosthesis within the branching implant site.

### RELATED APPLICATIONS

This application claims priority to United States Provisional Application No. 63/721,975, filed on November 18, 2024, and titled, “Apparatuses and Methods for Expandable Branching Stent Prosthesis,” which is hereby incorporated by reference in its entirety.

### TECHNICAL FIELD

The present disclosure relates to medical devices including stent prostheses, deployment systems, and methods of using the same.

### BRIEF DESCRIPTION OF THE SEVERAL VIEWS OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1A illustrates a front side view of an expandable branching stent prosthesis, according to embodiments described herein.

FIG. 1B illustrates the expandable branching stent prosthesis of FIG. 1A within a branching implant site, according to embodiments described herein.

FIG. 1C illustrates a deployment system configured for use with the expandable branching stent prosthesis of FIG. 1A, according to embodiments described herein.

FIG. 2 illustrates a cross sectional view of a proximal end of the deployment system of FIG. 1C, according to embodiments described herein.

FIG. 3A illustrates a deployment system configured for use with the expandable branching stent prosthesis of FIG. 1A, according to embodiments described herein.

FIG. 3B illustrates cross sectional view of a proximal end of the deployment system of FIG. 3A, taken through plane 3B-3B of FIG. 3A, according to embodiments described herein.

FIG. 4A illustrates a deployment system configured for use with expandable branching stent prosthesis of FIG. 1A, according to embodiments described herein.

FIG. 4B illustrates a protective portion of a balloon of the deployment system of FIG. 4A, according to embodiments described herein.

FIG. 5A illustrates an initial deployment stage of a deployment system, according to embodiments described herein.

FIG. 5B illustrates a distal deployment stage for the deployment system of FIG. 5A, according to embodiments herein.

FIG. 5C illustrates a joined deployment stage for the deployment system of FIG. 5A, according to embodiments herein.

FIG. 5D illustrates a deployment stage for an alternative deployment system, according to embodiments herein.

FIG. 5E illustrates a snaring deployment stage for the deployment system of FIG. 5A, according to embodiments described herein.

FIG. 5F illustrates a contralateral, sheath-docking stage for the deployment system of FIG. 5A, according to embodiments described herein.

FIG. 5G illustrates an ipsilateral, sheath-docking stage for the deployment system of FIG. 5A, according to embodiments described herein.

FIG. 5H illustrates a seating stage for the deployment system of FIG. 5A, according to embodiments described herein.

FIG. 5I illustrates a partially expanded stage for the deployment system of FIG. 5A, according to embodiments described herein.

FIG. 5J illustrates a partially expanded stage for the deployment system of FIG. 5A, according to embodiments described herein.

FIG. 5K illustrates a fully expanded stage for deployment system 522 of FIG. 5A, according to embodiments described herein.

FIG. 5L illustrates a post-deployment stage for the deployment system of FIG. 5A, according to embodiments described herein.

### DETAILED DESCRIPTION

Delivery catheter systems may be configured to deliver one or more medical appliances or systems to a location within a patient’s body and deploy the medical appliance or system within the patient’s body. For example, such a delivery catheter system may be configured to be advanced from an insertion site at the outside of an anatomical system to a treatment location within the anatomical system. For example, a delivery catheter system may be configured to be advanced through bends, turns, or other structures within the anatomy of the vasculature.

A stent prosthesis may be disposed within a portion of the delivery catheter system (e.g., as or as part of a medical appliance or system) such that a practitioner may deploy the stent prosthesis from a distal end of the delivery catheter system through manipulation of one or more components of a handle assembly of the delivery catheter system.

Stent prostheses may be deployed in various body lumens for a variety of purposes. Stent prostheses may be deployed, for example, in the arterial system for a variety of therapeutic purposes including the treatment of occlusions within the lumens of that anatomical system. It will be appreciated that the current disclosure may be applicable to stent prostheses designed for the central venous system, peripheral vascular system, abdominal aortic aneurism treatment, bronchial system, esophageal system, biliary system, or any other system of the human body. Further, the present disclosure may equally be applicable to other prosthesis such as grafts.

Accordingly, it will be understood that while specific examples recited herein may refer to deployment of cardiovascular stent prostheses within a cardiovascular system, analogous concepts and devices may be used in/with various other anatomical systems of the body, including for placement and deployment of medical appliances in the gastrointestinal tract (including, for example, within the esophagus, intestines, stomach, small bowel, colon, and biliary duct); the respiratory system (including, for example, within the trachea, bronchial tubes, lungs, nasal passages, and sinuses); or any other location within the body, both within bodily lumens (for example, the ureter, the urethra, etc.) and within other bodily structures.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “proximal” and “distal” are used herein to refer to opposite locations relative to a medical device in use by a practitioner. The proximal end of the device is defined as the end of the device closest to the practitioner when the device is in use by the practitioner. The distal end is the end opposite the proximal end.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

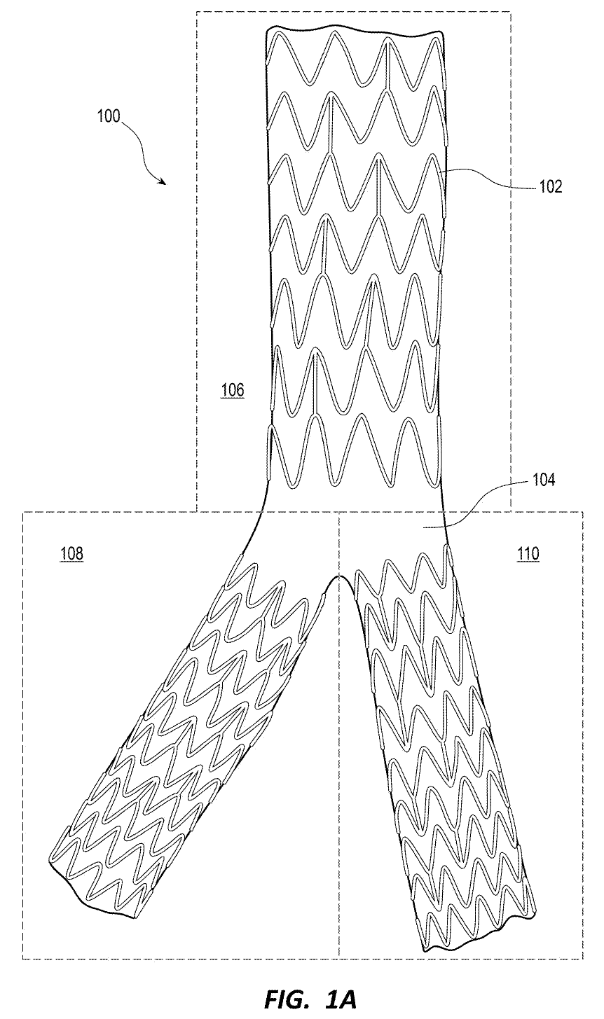

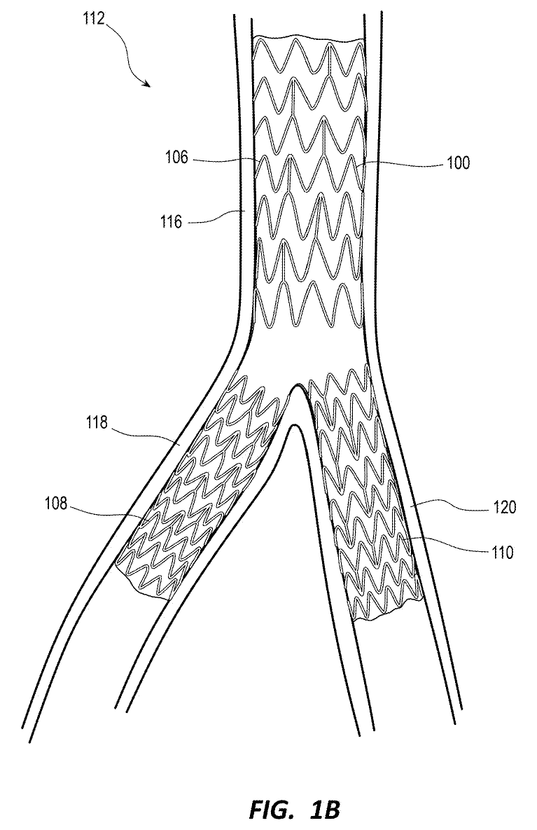

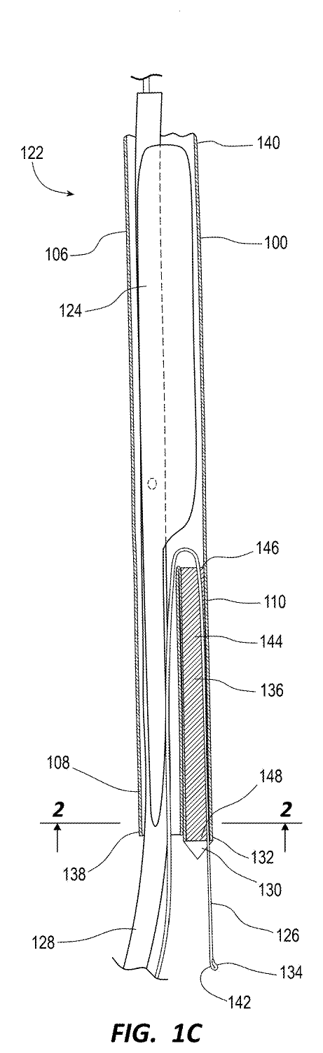

FIG. 1A illustrates a front side view of an expandable branching stent prosthesis 100, according to embodiments described herein. Expandable branching stent prosthesis 100 of FIG. 1A is illustrated in a deployed (expanded) position.

In the illustrated embodiment, expandable branching stent prosthesis 100 is partially composed of a wire or laser cut stent 102. The wire or laser cut stent 102 is configured to be expandable to the illustrated deployed position from a collapsed/unexpanded/un-deployed configuration (e.g., via the inflation of one or more balloons from within expandable branching stent prosthesis 100) after expandable branching stent prosthesis 100 is delivered to a desired location. The wire or laser cut stent 102 may be formed of any suitable material, such as nickel-titanium alloy, stainless steel, cobalt-chromium, platinum, polymers, etc. The wire or laser cut stent 102 may have a zig-zag pattern, a wave pattern, or any other suitable pattern. The wire or laser cut stent 102 may be pre-formed or formed corresponding to tubular body 104. The material, pattern, and wire diameter of a wire or laser cut stent 102 that is a wire stent, or the wall thickness and strut width of a wire or laser cut stent 102 that is a laser cut stent, may be configured to provide a chronic radial outwardly directed force and a resistance to a radial inwardly directed force. For a non-self-expanding design, the deployed wire or laser cut stent 102 may be configured to provide a radial stiffness and radial strength to resist a local or radial inwardly directed force.

Expandable branching stent prosthesis 100 further includes a tubular body 104. Tubular body 104 may be formed of a variety of materials and/or layers of materials, including biocompatible materials that are resistant to passage of fluid through a wall of tubular body 104. For example, tubular body 104 may be formed of polyethylene terephthalate, polyurethane, silicone rubber, nylon, fluoropolymer, polyester, etc. A thickness of the wall may range from about 0.025 mm to about 0.5 mm.

In certain embodiments, the wall of tubular body 104 may be impermeable to tissue cell ingrowth into and/or tissue cell migration through the wall, for example, to prevent or discourage stenosis of tubular body 104. Additionally, or alternatively, in some embodiments, the wall of tubular body 104 can be impermeable to fluid such that fluid is prevented from leaking from the inside of expandable branching stent prosthesis 100 to the exterior of expandable branching stent prosthesis 100 and into surrounding tissue. In some embodiments an interior surface of the wall may include serially deposited fibers of polytetrafluoroethylene (PTFE) to resist fibrin deposition and platelet adhesion on the surfaces.

Note that in embodiments herein, stent prostheses are illustrated as having tubular bodies (such as tubular body 104 of FIG. 1A). However, it will be understood that expandable branching stent prostheses using a wire scaffold, framework, or stent without a cover or other tubular body fall within the scope of the disclosure.

As illustrated herein, expandable branching stent prosthesis 100 may be for deployment at a branching implant site within a body having a trunk, a first branch, and a second branch. Accordingly, expandable branching stent prosthesis 100 includes a trunk portion 106, a first branching portion 108, and a second branching portion 110. For deployment of expandable branching stent prosthesis 100 at the branching implant site, trunk portion 106 of expandable branching stent prosthesis 100 is used in/at the trunk of the branching implant site, first branching portion 108 is deployed in/at the first branch of the branching implant site, and second branching portion 110 is deployed in/at the second branch of the branching implant site.

FIG. 1B illustrates expandable branching stent prosthesis 100 of FIG. 1A within a branching implant site 112, according to embodiments described herein. In this expanded/deployed configuration, expandable branching stent prosthesis 100 provides stenting at/through branching implant site 112 within the anatomical system in which expandable branching stent prosthesis 100 is deployed.

Branching implant site 112 can be a portion of an anatomical system that includes a first branch 118, a second branch 120, and a trunk 116 (and that, e.g., communicates fluids through these). In some cases, branching implant site 112 may be the aorto-iliac bifurcation in the arterial system. In the illustrated deployed configuration, trunk portion 106 of expandable branching stent prosthesis 100 is deployed at/within trunk 116 of branching implant site 112, first branching portion 108 of expandable branching stent prosthesis 100 is deployed at/within first branch 118 of branching implant site 112, and second branching portion 110 of expandable branching stent prosthesis 100 is deployed at and/or within second branch 120 of branching implant site 112.

In some embodiments, expandable branching stent prosthesis 100, once deployed, provides an appropriate channel for desired liquid flow through branching implant site 112. It may be that the region around branching implant site 112 is diseased, misshapen, and/or damaged, and that the deployment of expandable branching stent prosthesis 100 in the illustrated manner can correct and/or ameliorate attendant issues. For ease of illustration and description, the figures do not illustrate branching implant site 112 as diseased, misshapen, and/or damaged.

As illustrated, branching implant site 112 of FIG. 1B is used within an anatomical system that is a cardiovascular system. However, it should be noted that while examples herein describe (and figures herein illustrate) the deployment of expandable branching stent prostheses within cardiovascular systems, the cardiovascular anatomical context is given by way of example and not by way of limitation. It will be understood that deployment systems analogous to those described in relation to disclosure herein may be used to deploy appropriate expandable branching stent prostheses with/at branching implant sites of other anatomical systems, and that corresponding methods for using such deployment systems to deploy corresponding expandable branching stents that are analogous to those methods described herein could be used in those other anatomical contexts.

FIG. 1C illustrates a deployment system 122 configured for use with expandable branching stent prosthesis 100 of FIG. 1A, according to embodiments described herein. As illustrated, deployment system 122 may include expandable branching stent prosthesis 100, a balloon 124, and a floss wire 126. Furthermore, expandable branching stent prosthesis 100 is illustrated in FIG. 1C in a collapsed (unexpanded, compressed, non-deployed, etc.) configuration, but is otherwise as described in relation to FIG. 1A. Otherwise stated, expandable branching stent prosthesis 100 as seen in FIG. 1C can be configured to expand (into an expanded configuration) from the unexpanded configuration as illustrated.

Deployment system 122 may be mounted on a catheter 128. Deployment system 122 can be mounted by passing catheter 128 through first branching portion 108 and trunk portion 106 of expandable branching stent prosthesis 100. In the illustrated embodiment, the unexpanded configuration of expandable branching stent prosthesis 100 can be seen.

Balloon 124 can be arranged within expandable branching stent prosthesis 100. For instance, in embodiments, balloon 124 may be attached to a catheter lumen 129 of the catheter 128 along its length and may be inflated and/or deflated at the option of a practitioner operating a catheter delivery system (not shown) via a connection 127 between balloon 124 and catheter lumen 129 that communicates fluid between catheter lumen 129 and balloon 124. Balloon 124 can extend from a proximal end 138 of first branching portion 108, to a distal end 140 of trunk portion 106. As can be seen, due to the shape of balloon 124 and its placement/arrangement within expandable branching stent prosthesis 100, when balloon 124 is inflated balloon 124 expands trunk portion 106 and first branching portion 108 of expandable branching stent prosthesis 100 for deployment.

A distal end 134 of floss wire 126 passes through first branching portion 108 of expandable branching stent prosthesis 100 and out second branching portion 110 of expandable branching stent prosthesis 100, as illustrated. Distal end 134 of floss wire 126 passes along the outside of balloon 124 in such a manner that there is substantially no translational dependency between floss wire 126 and balloon 124. For instance, in embodiments, floss wire 126 may be advanced or retracted through expandable branching stent prosthesis 100 without necessarily repositioning balloon 124. Distal end 134 of floss wire 126 terminates in a catch feature 142 that is configured to interact with a snare feature of a snare wire, in the manner described herein. Catch feature 142 of floss wire 126 can be disposed some distance from a distal end 132 of second branching portion 110.

When deployed within an anatomical system, floss wire 126 may extend back through a branch of a branching implant site for planned deployment of expandable branching stent prosthesis 100 of deployment system 122 and run to/through an insertion site through which a practitioner has delivered deployment system 122 to the body (e.g., using a delivery system (such as, e.g., a delivery catheter system) that includes deployment system 122.

In the unexpanded configuration, second branching portion 110 of deployment system 122 can include a fluid-conductive lumen 144. Fluid-conductive lumen 144 may be formed by compressing second branching portion 110 onto a mandrel during manufacture that is removed after second branching portion 110 is collapsed. Otherwise stated, when in the unexpanded configuration with mandrel removed, second branching portion 110 can include fluid-conductive lumen 144. Within the unexpanded, or collapsed configuration, fluid-conductive lumen 144 can include a proximal end 146 which is closed to the passage of fluid. In other words, proximal end 146 of fluid-conductive lumen 144 terminates at a collapsed state of trunk portion 106. A distal end 148 of lumen 144 (and distal end 132 of second branching portion 110) can be open to the passage of fluid in the unexpanded or collapsed configuration.

In some embodiments, floss wire 126 can be, or include, an enlarged portion that functions as a mandrel to form fluid-conductive lumen 144. The enlarged portion of floss wire 126 can define fluid-conductive lumen 144 within second branching portion 110 when second branching portion 110 is compressed during manufacture. After second branching portion 110 is compressed, the enlarged portion of the floss wire 126 is pulled out of the fluid-conductive lumen 144 leaving a smaller diameter portion of floss wire 126 in the fluid-conductive lumen 144. Otherwise stated, fluid-conductive lumen 144 of second branching portion 110, can be formed when the enlarged portion of floss wire 126 is removed from within second branching portion 110.

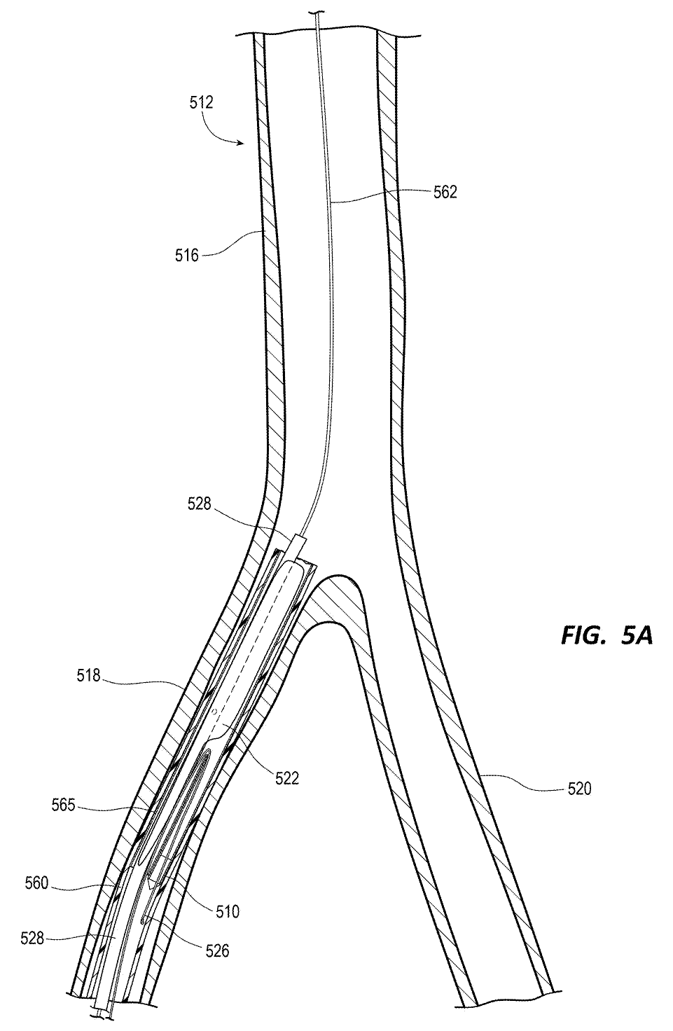

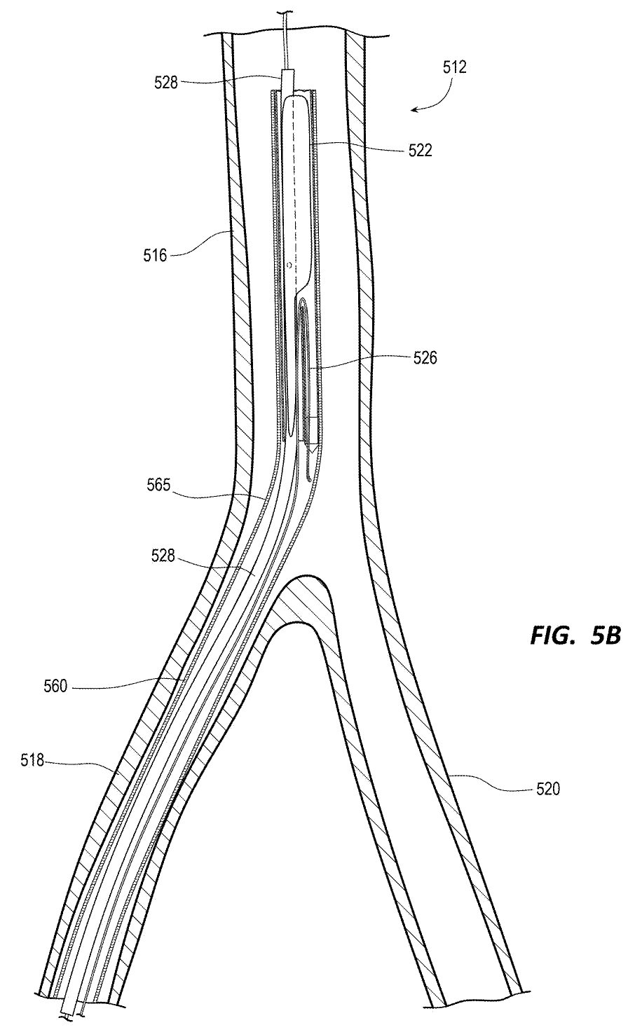

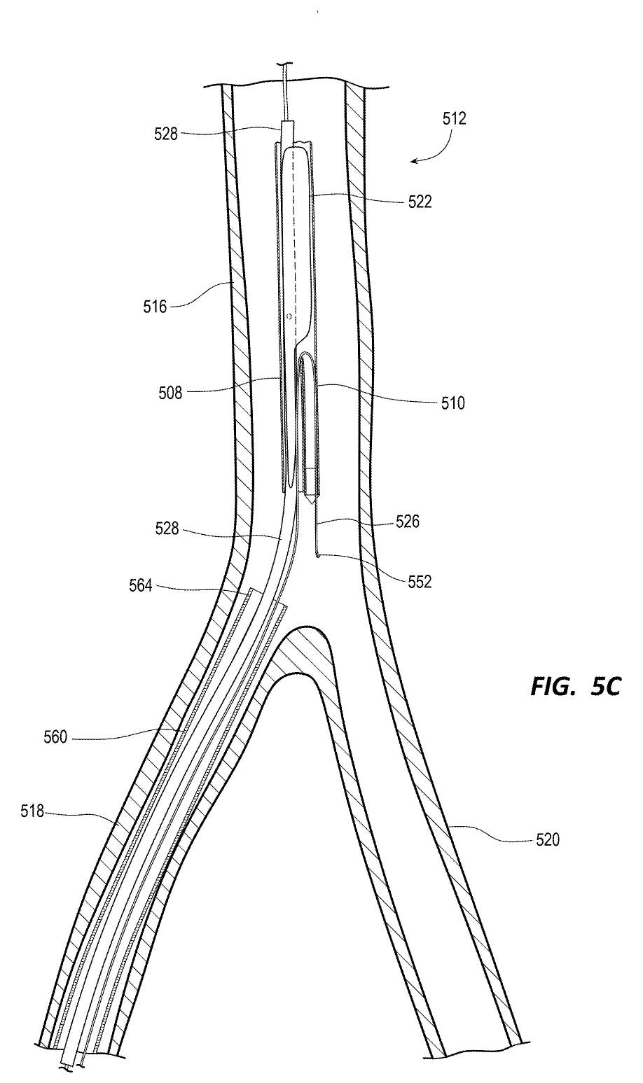

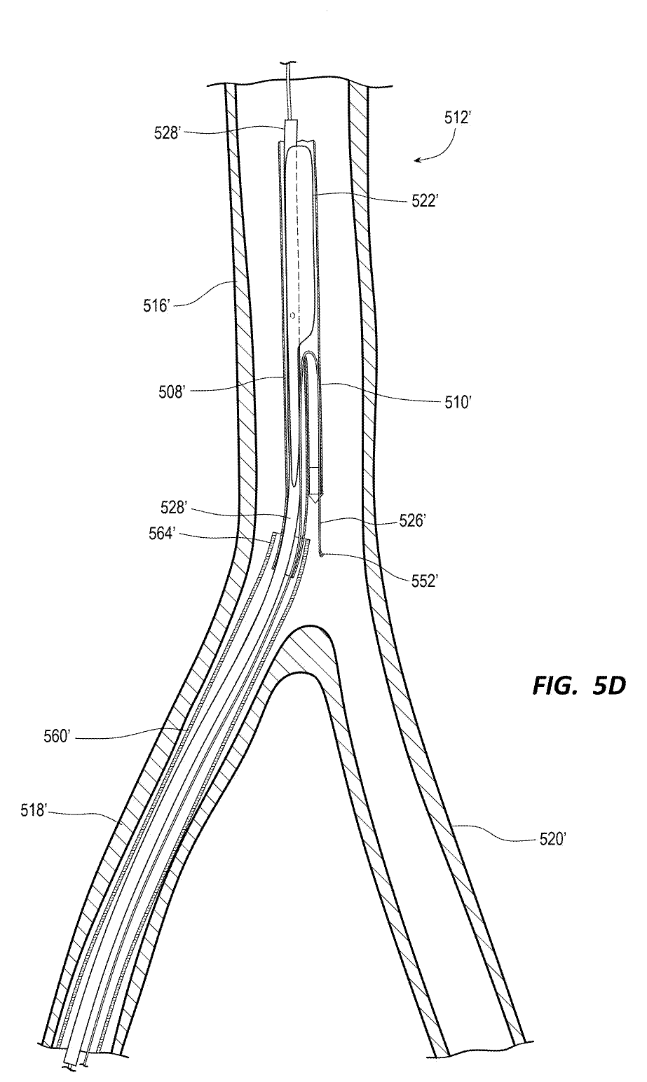

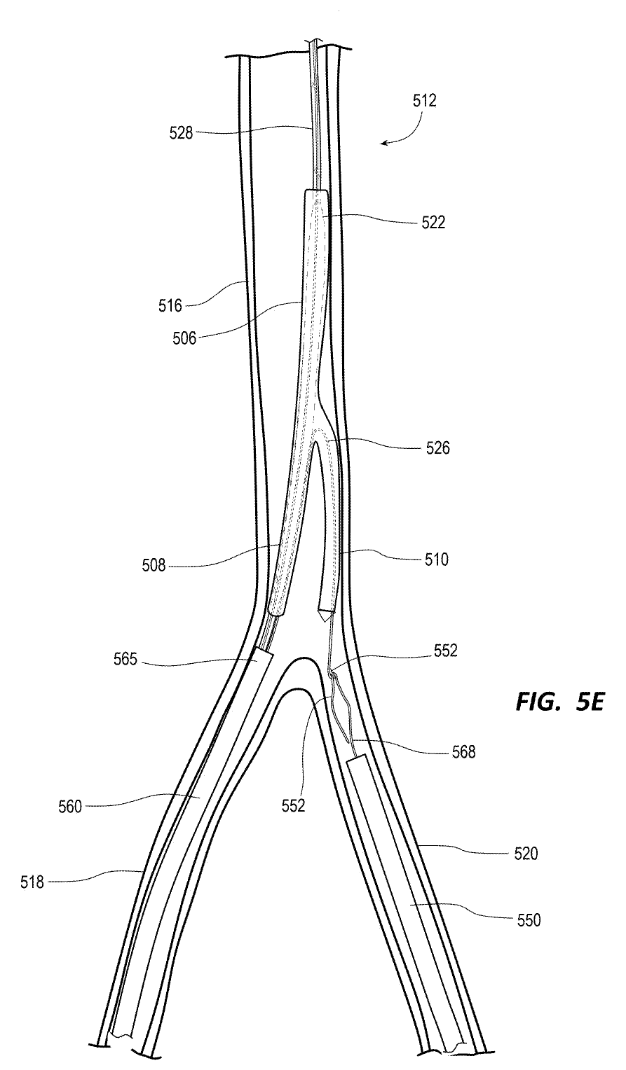

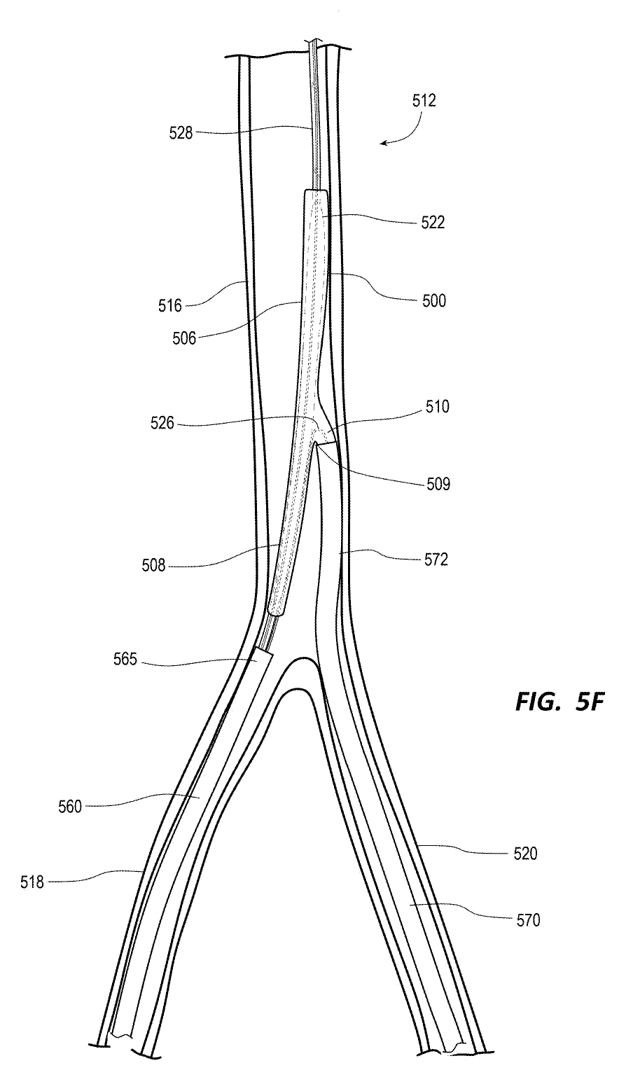

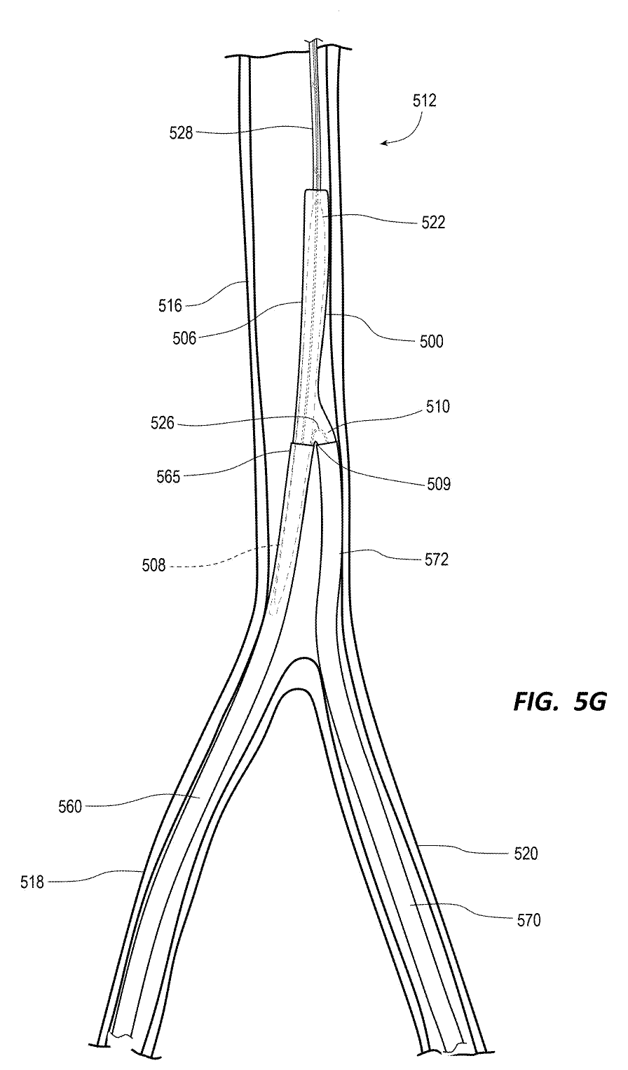

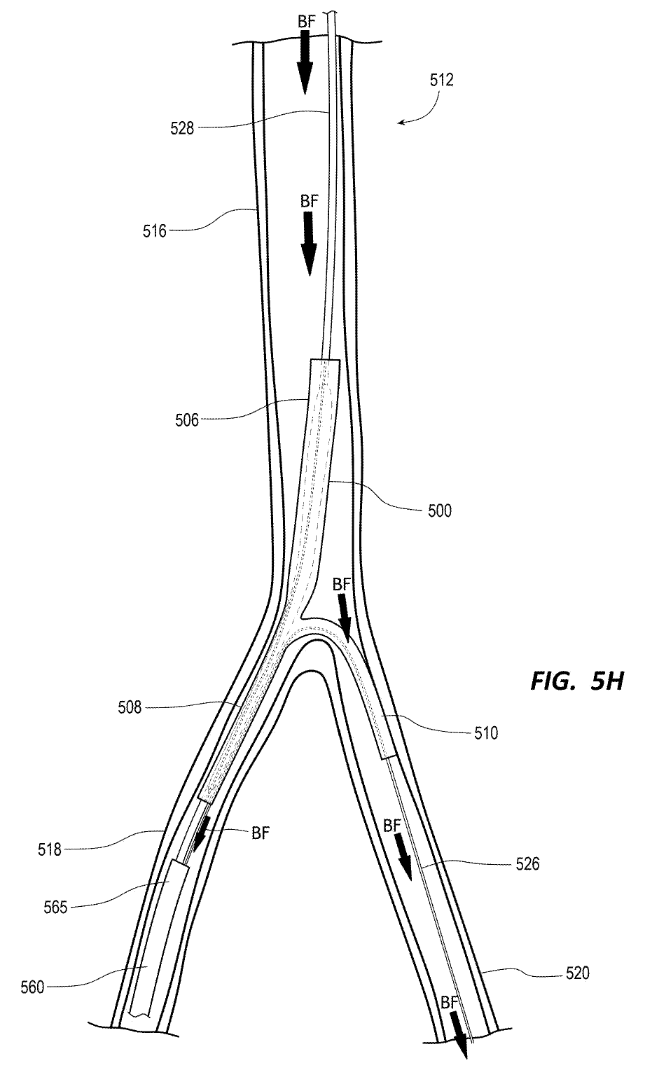

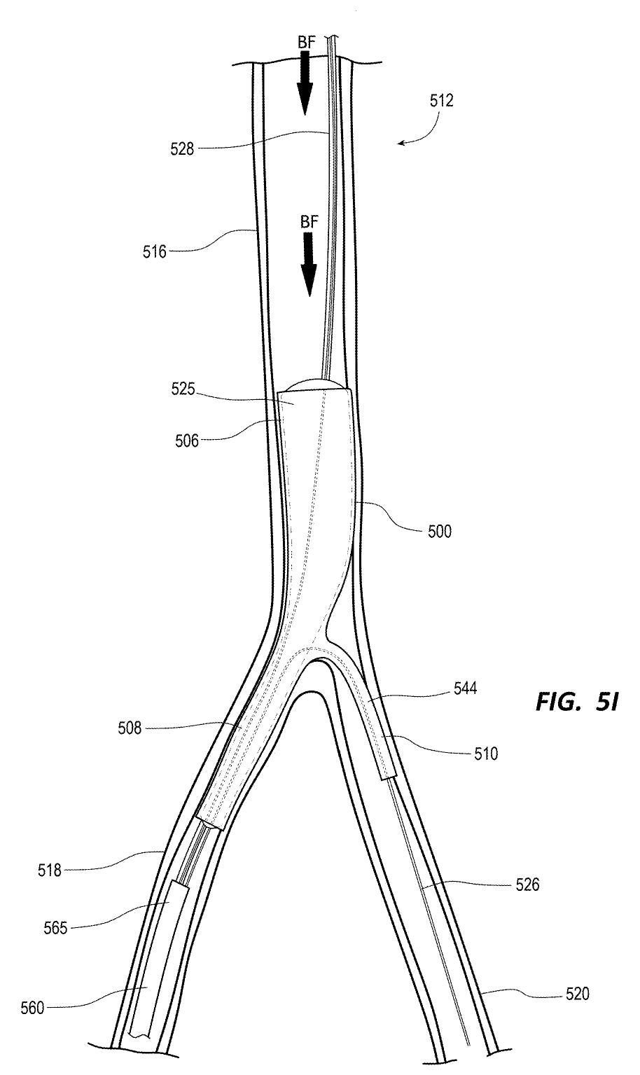

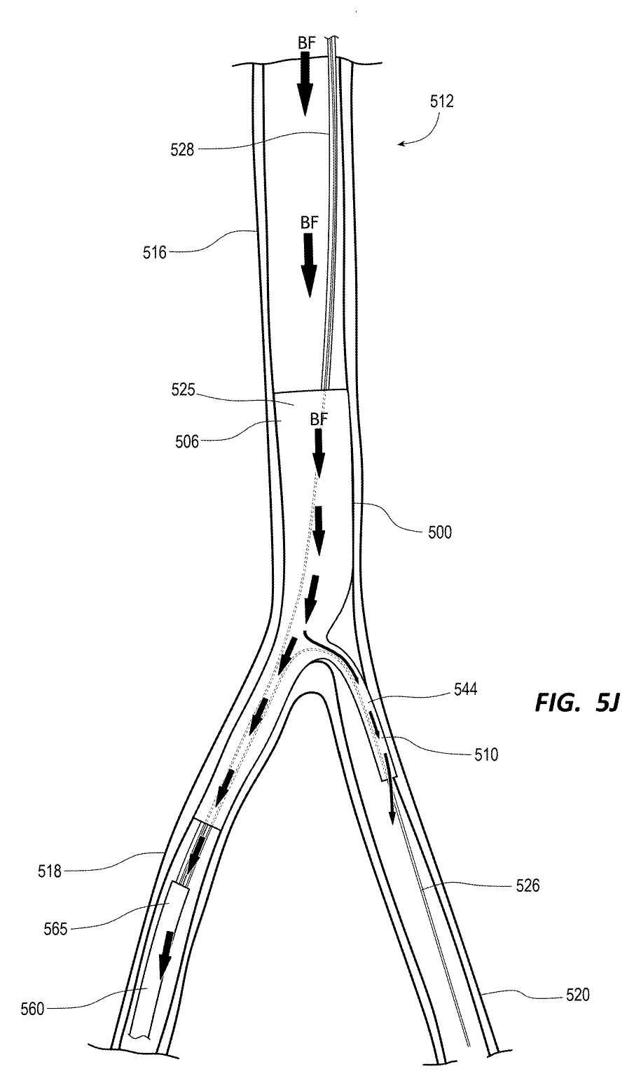

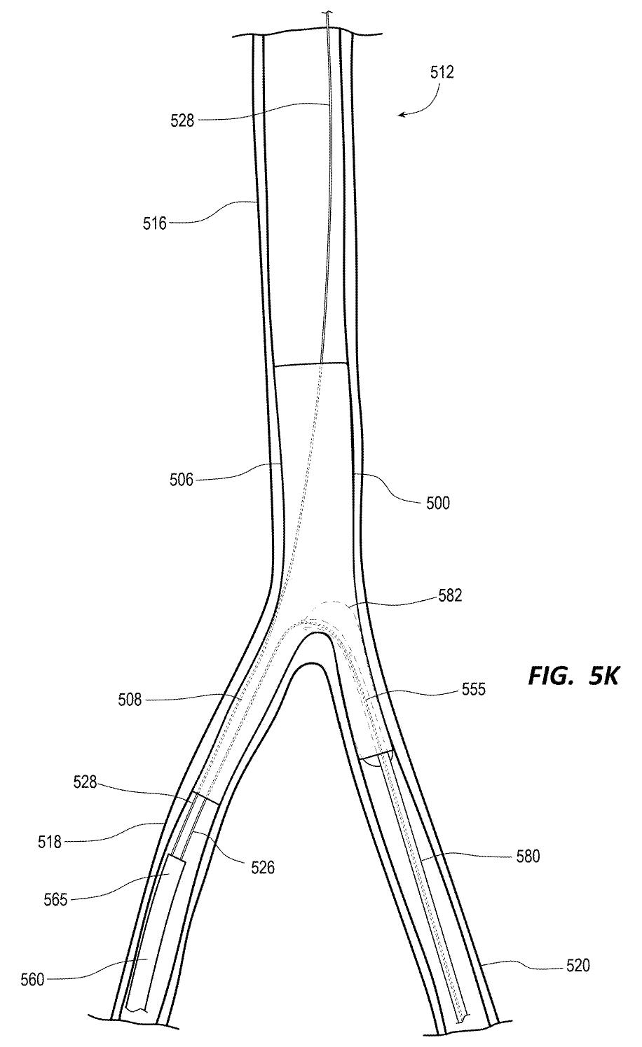

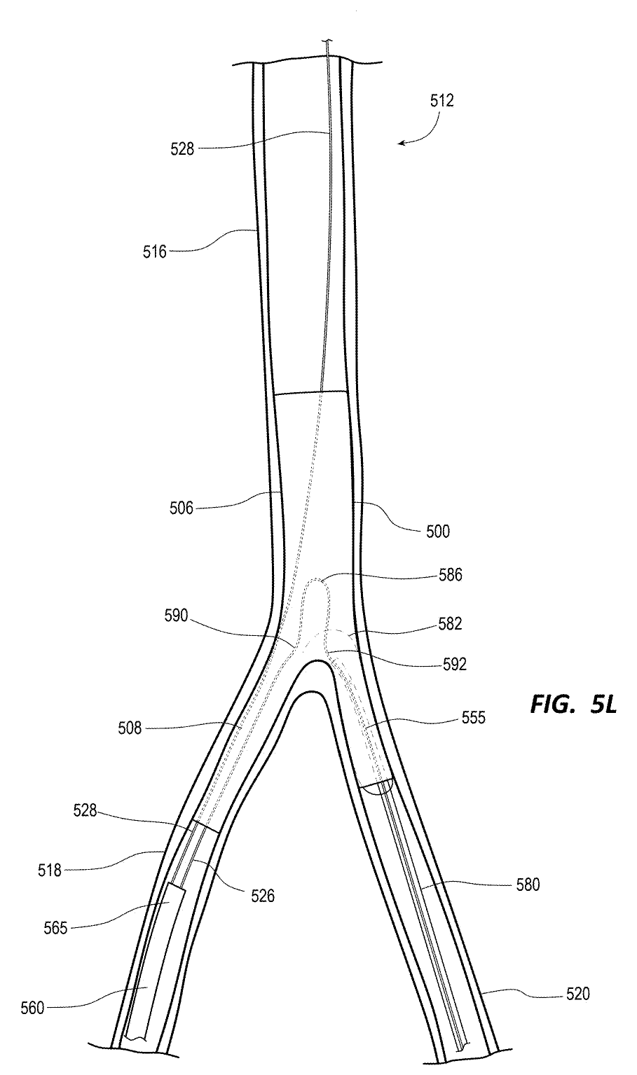

As will be further described with respect to FIGS. 5A-5K, when expandable branching stent prosthesis 100 is placed within the vascular branching implant site for deployment, expandable branching stent prosthesis 100 can be expanded in stages. In an initial, partially expanded stage or configuration, trunk portion 106 and first branching portion 108 can be expanded, prior to the expansion of second branching portion 110. In the partially expanded configuration, expansion of trunk portion 106 and first branching portion 108 may form fluid-conductive lumens through each expanded portion. Otherwise stated, fluid (e.g., blood), may flow through the interior of trunk portion 106 and first branching portion 108 after each portion has been expanded.

In the partially expanded configuration, expansion of trunk portion 106 can transition proximal end 146 of fluid-conductive lumen 144 from a closed configuration to an open configuration. Thus, fluid can be permitted to flow from a lumen of trunk portion 106 in an expanded state, through second branching portion 110, and into second branch, when second branching portion 110 is in an unexpanded state.

As discussed above, fluid-conductive lumen 144 can be formed by inserting mandrel into deployment system 122 during crimping, or collapsing, of stent prosthesis 100 into the unexpanded state. In some embodiments, after stent prosthesis 100 has been crimped or collapsed, mandrel can be removed from deployment system 122, and crimped stent prosthesis 100. Removal of mandrel can leave a void or cavity or fluid-conductive lumen 144 within second branching portion 110. This void or cavity can form fluid-conductive lumen 144.

As discussed above, mandrel can be, or include, an expanded portion, integral to floss wire 126. For instance, mandrel can be an expanded portion of floss wire 126 that enlarges to fit within an inner circumference of the unexpanded second branching portion 110. In embodiments, where floss wire 126 comprises an enlarged portion that serves as mandrel, mandrel can stay disposed within deployment system 122 as it is deployed in the branching implant site. In other embodiments, mandrel can be disposed within deployment system 122 during manufacturing, and removed during a later manufacturing process, or prior to insertion.

Absence of a similar mandrel within first branching portion 108 and trunk portion 106 during crimping, or collapsing, can indicate that a lumen is not formed within those portions. Thus, when in an unexpanded state, first branching portion 108 and trunk portion 106 may not contain a fluid-conductive lumen in the crimped state.

In the illustrated embodiment, deployment system 122 may further include a protective shroud 130 that can be disposed adjacent to distal end 148 of fluid-conductive lumen 144 and distal end 132 of second branching portion 110. In some embodiments, protective shroud 130 is disposed exteriorly adjacent to a longitudinal end of second branching portion 110. Protective shroud 130 can be distally adjacent, or slightly spaced, from distal end 132. Protective shroud 130 can include a flared, or tapered, outer portion. In embodiments, protective shroud 130 can be a substantially conical shape.

When expandable branching stent prosthesis 100 is in the unexpanded, or crimped configuration, an outer edge, or portion, of protective shroud 130 can extend radially further than the wall of second branching portion 110. Otherwise stated, an overall diameter of protective shroud 130 can be larger than or exceed an overall diameter of second branching portion 110 in the unexpanded state.

As will be further described in connection with FIG. 5E, the larger diameter of protective shroud 130 can protect distal end 132 of second branching portion 110, when a sheath is slid over second branching portion 110. The conical shape, or profile, of protective shroud 130 can also serve to guide a sheath as it is slid axially over second branching portion 110 to prevent the sheath from getting caught on distal end 132 of expandable branching stent prosthesis 100. In some embodiments, protective shroud 130 may prevent distal end 132 from catching on a vessel wall or vessel plaque while second branching portion 110 is seated into second branch 120. In some embodiments, protective shroud 130 acts as a shield to the longitudinal end of second branching portion 110.

Similar to a mandrel, in embodiments, protective shroud 130 can be, or include, an expanded portion, integral to floss wire 126. For instance, protective shroud 130 can be an expanded portion of floss wire 126, which enlarges to extend radially past an outer circumference of unexpanded second branching portion 110. In embodiments where floss wire 126 comprises an enlarged portion that serves as protective shroud 130, protective shroud 130 can be disposed within deployment system 122 as it is deployed in the branching implant site.

In some embodiments, the protective shroud 130 may include a cylindrical portion 136 that extends into the fluid-conductive lumen 144.

While the protective shroud 130 is discussed in regard to expandable branching stent prosthesis 100, the use of the protective shroud 130 is not so limited. The protective shroud 130 may be used with cylindrical stents and the like.

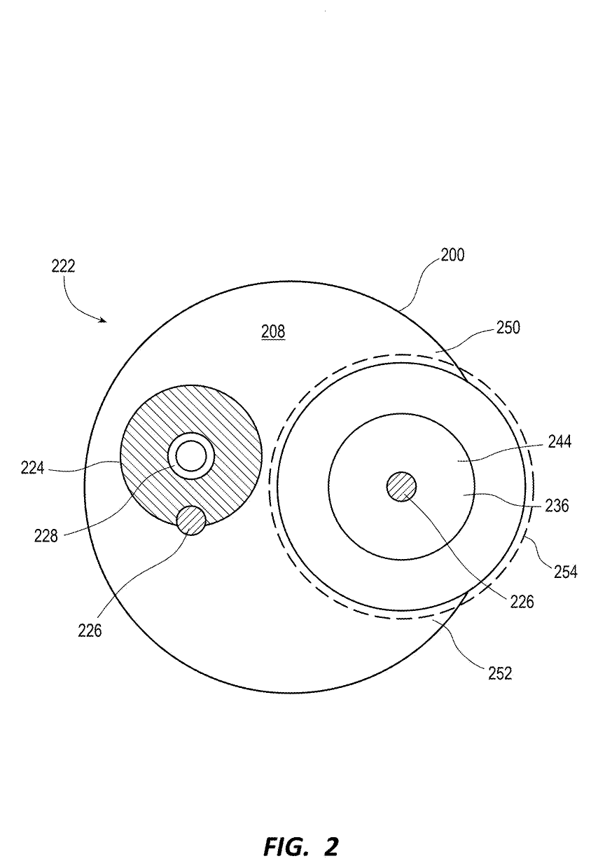

FIGS. 2, 3A-3B, 4A-4B and 5A-5K illustrate embodiments of stent prosthesis (and corresponding deployment systems) that resemble the stent prosthesis (and corresponding deployment systems) described with respect to FIGS. 1A-1C, in certain respects. Accordingly, like features are designated with like reference numerals. For example, the embodiments illustrated in FIGS. 2, 3A-3B, 4A-4B and 5A-5K include a floss wire 226, 326, 526 that may, in some respects, resemble floss wire 126 of FIGS. 1A-1C. Relevant disclosure set forth above regarding similarly identified features thus may not be repeated hereafter. Moreover, specific features of the expandable branching stent prosthesis and corresponding deployment systems of FIGS. 2, 3A-3B, 4A-4B and 5A-5K and related components shown in FIGS. 2, 3A-3B, 4A-4B and 5A-5K, may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiments of the stent prosthesis and corresponding deployment systems and related components illustrated in FIGS. 2, 3A-3B, 4A-4B and 5A-5K. Any suitable combination of the features, and variations of the same, described with respect to the stent prosthesis, corresponding deployment systems, and related components described with respect to FIGS. 1A-1C can be employed with the embodiments the stent prosthesis, corresponding deployment systems, and related components of FIGS. 2, 3A-3B, 4A-4B and 5A-5K, and vice versa. This pattern of disclosure applies equally to further embodiments depicted in subsequent figures and described hereafter, wherein the leading digits may be further incremented.

FIG. 2 illustrates a cross sectional view (e.g., taken from the analogous location of plane 2-2 of FIG. 1C), of proximal end 138 of a deployment system similar to the deployment system 122 of FIG. 1C, according to embodiments described herein. In the illustrated embodiment, first branching portion 208 and second branching portion 210 of expandable branching stent prosthesis 200 can be seen in an unexpanded configuration. As compared to the embodiment of FIG. 1C, first branching portion 208 can secure second branching portion 210 such that a combined profile of first branching portion 208 and second branching portion 210 is substantially circular (or substantially circular-shaped.

The proximal end of deployment system 222 can include first branching portion 208 in an unexpanded, or crimped state. First branching portion 208 can be crimped or collapsed around floss wire 226, balloon 224, and catheter 228.

Second branching portion 210 can be seen in an unexpanded, or crimped state. Second branching portion 210 has a circular profile. Second branching portion 210 can be crimped or collapsed around floss wire 226. As discussed with respect to FIG. 1C, second branching portion 210 can include fluid-conductive lumen 244 formed by the mandrel during the manufacturing process. For simplicity, the protective shroud is not shown with respect to FIG. 2, although the outer perimeter or diameter 254 of the protective shroud is designated via a dashed line.

As seen, first branching portion 208 can be configured in a semi-circular, or crescent shaped profile. In such a state, outer ends 250, 252 if the crescent shaped profile of first branching portion 208 can substantially envelop and/or secure second branching portion 210. In embodiments, first branching portion 208 can be crimped tightly around second branching portion 210, which can be disposed with a substantially circular profile. As illustrated in FIG. 2, second branching portion 210 may not be completely enveloped by first branching portion 208 and a portion of second branching portion 210 may extend laterally outward, giving the substantially circular profile a slight lateral bump.

First branching portion 208 can secure second branching portion 210 such that a threshold force is required that is sufficient to remove second branching portion 210 from securement of first branching portion 208. For instance, a lateral, removal, force can be applied to second branching portion 210 through floss wire 226. Once the threshold force is applied, second branching portion 210 can disengage, or dislodge, from securement from first branching portion 208. Second branching portion 210 can maintain a substantially circular profile throughout securement and dislodgement from first branching portion 208.

In the illustrated embodiments, first branching portion 208 can be compressed to envelop second branching portion 210 of expandable branching stent prosthesis 200. For example, first branching portion 208 can envelop second branching portion 210 through a profile that is substantially crescent-shaped, curved, lunar, or semi-circular. For example, stent prosthesis 200 can be compressed such that ends 250, 252 of first branching portion 208 encircle second branching portion 210. Otherwise stated, second branching portion 210 can be configured to fit within a curved profile of first branching portion 208. Second branching portion 210 can have a substantially circular profile.

Ends 250, 252 of first branching portion 208 can retain second branching portion 210. An outer diameter 254 of a protective shroud can extend radially further than an outer diameter of second branching portion 210.

As will be further described with respect to FIGS. 5C-5D, the securement of second branching portion 210 can be disengaged from envelopment, such that first branching portion 208 and second branching portion 210 become separated. Otherwise stated, in some cases, second branching portion 210 can become unsecured, or disengaged, from first branching portion 208. In some cases, a minimum threshold force can be required to detach, disengage, or unsecure, second branching portion 210 from retaining by first branching portion 208.

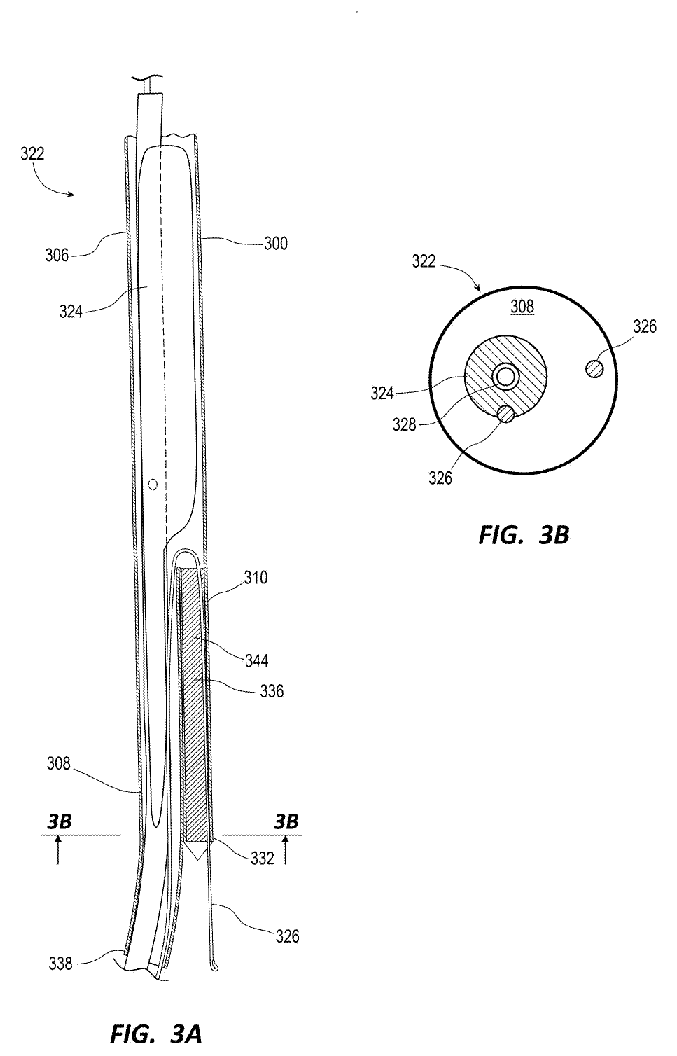

FIG. 3A illustrates deployment system 322 configured for use with expandable branching stent prosthesis 300, similar in some respects to expandable branching stent prosthesis 100 of FIG. 1A, according to embodiments described herein. Expandable branching stent prosthesis 300 includes trunk portion 306, first branching portion 308, and second branching portion 310. As compared to the embodiment of FIG. 1C, proximal end 338 of first branching portion 308 extends proximally further than proximal end 332 of second branching portion 310. Otherwise stated, proximal end 338 of first branching portion 308 can extend, or be longer, than second branching portion 310. Otherwise stated, proximal end 338 of first branching portion 308 can extend beyond a free end of second branching portion 310.

FIG. 3B illustrates cross sectional view, taken through plane 3B-3B of FIG. 3A, of proximal end of the deployment system 322 of FIG. 3A, according to embodiments described herein. As seen in the illustrated embodiment, the proximal end of deployment system 322 can include first branching portion 308 in an unexpanded, or crimped state. In the illustrated embodiment, floss wire 326 is free from first branching portion 208. In some embodiments, first branching portion 308 can be crimped or collapsed around floss wire 326, balloon 324, and catheter 328.

In some embodiments, first branching portion 308 can continue to secure second branching portion 310 (as was seen with respect to FIG. 2). However, a longer ipsilateral portion of first branching portion 308 can continue to extend, such that the proximal end profile of first branching portion 308 is substantially circular. For instance, second branching portion 310 can terminate, and first branching portion 308 can continue. Otherwise stated, the cross-sectional profile of deployment system 322 can transition from the profile seen in FIG. 2, to the profile seen in FIG. 3B. Otherwise stated, second branching portion 310 can end, and first branching portion 308 can continue proximally, such that the proximal end profile is substantially circular. In embodiments, this can aid in sheathing, or sliding a protective cover or sheath over proximal end 338 of first branching portion 308.

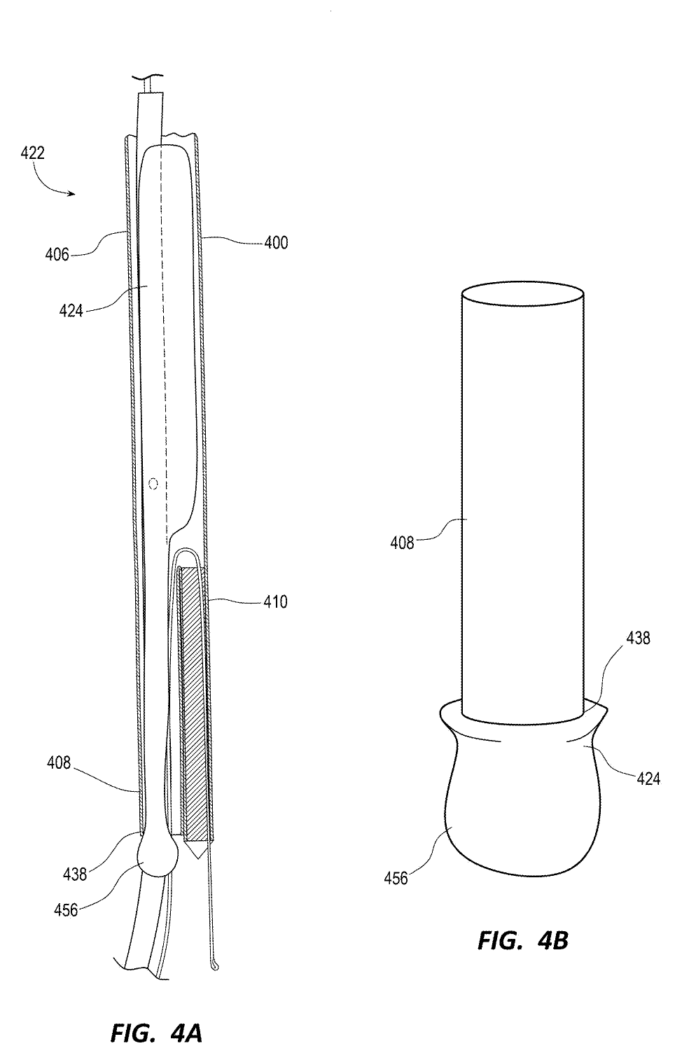

FIG. 4A illustrates a deployment system 422 configured for use with expandable branching stent prosthesis 400, similar in some respects to expandable branching stent prosthesis 100 of FIG. 1A, according to embodiments described herein. Expandable branching stent prosthesis 400 includes trunk portion 406, first branching portion 408, and second branching portion 410. As compared to the embodiment of FIG. 1C, balloon 424 may comprise a protective portion 456 that can extend proximally further than first branching portion 408. Otherwise stated, protective portion 456 of balloon 424 can extend proximally out of distal end 438 of first branching portion 408.

FIG. 4B illustrates a perspective view of protective portion 456 of balloon 424 of FIG. 4A, according to embodiments described herein. In some embodiments, protective portion 456 of balloon 424 can fold, or pillow around an exterior circumference of distal end 438 of first branching portion 408, and protect distal end 438 (e.g., in an analogous manner to protective shroud 130, as was discussed with respect to FIG. 1C). For example, when a sheath is pushed, or advanced, over, or around distal end 438, protective portion 456 can fold, or pillow, over and around distal end 438, thus protecting distal end 438 of first branching portion 408.

FIGS. 5A–5K illustrate a method of deploying an expandable branching stent prosthesis 500 at a branching implant site 512. FIG. 5A illustrates an initial deployment stage of deployment system 522, similar in some respects to deployment system 122 of FIG. 1C, according to embodiments described herein. A catheter delivery system 560 may be used to deliver deployment system 522 to a desired location. The disclosure recited in connection with FIGS. 5A-5K may be applied to the deployment devices 122, 222, 322, 422 discussed above. In other words, any of the deployment devices 122, 222, 322, 422 discussed above may be used in connection with deployment procedures analogous to those of FIGS. 5A-5K. Certain variations to the process correlating to specific deployment devices are outlined below.

As discussed, deployment system 522 may be mounted on catheter 528 of catheter delivery system 560 by passing catheter 528 through first branching portion 508 and trunk portion 506 of expandable branching stent prosthesis 500. Catheter sleeve 564 of delivery system 560 may initially cover/surround deployment system 522 in order to hold deployment system 522 in place on catheter 528, and to prevent snagging of deployment system 522 during placement. Deployment system 522 can be tracked along a guidewire 562 of catheter delivery system 560. Once deployment system 522 has been located in the desired location, catheter sleeve 564 may be retracted back along catheter 528 to expose deployment system 522.

In alternative cases, it may be that deployment system 522 is not covered by catheter sleeve 564 during placement. In such a case, catheter 528 on which deployment system 522 is mounted (e.g., via crimping) would be manipulated to extend outward from catheter delivery system 560 until catheter 528 is located at the desired location.

One or more incisions (not illustrated) giving access to first branch 518 of branching implant site 512 is made in the body. Then, catheter delivery system 560 is used to deliver, via first branch 518, deployment system 522 to the illustrated location past first branch 518 and into trunk 516 of branching implant site 512.

While advancing up through first branch 518, deployment system 522 may be covered by catheter sleeve 564 of catheter delivery system 560 to facilitate positioning within trunk 516. Once the desired positioning of deployment system 522 is achieved, catheter sleeve 564 may be retracted along catheter 528 to expose deployment system 522 at a selected position e.g., such that the positioning of deployment system 522 is as illustrated in FIG. 5C (but prior to the use of a snare wire to snare floss wire 526, to be described below). Alternatively, catheter 528 may be advanced forward from catheter delivery system 560 until deployment system 522 is located in the desired position.

FIG. 5B illustrates a distal deployment stage for deployment system 522 of FIG. 5A, according to embodiments herein. In the illustrated embodiment, deployment system 522 can be seen as it has reached a distal distance into trunk 516 of branching implant site 512, and is ready for deployment. In some embodiments, this position may be a maximum distal distance needed to deploy the expanding branching stent prosthesis 500 in branching implant site 512.

FIG. 5C illustrates a joined deployment stage for deployment system 522 of FIG. 5A, according to embodiments herein. In the illustrated embodiment, deployment system 522 can be seen as it has reached trunk 516 of branching implant site 512, and catheter sleeve 564 has been retracted a distance along catheter 528.

As was discussed with respect to FIGS. 1C-4B, once deployment system 522 has reached the appropriate distance into the trunk, catheter sleeve 564 of deployment system 522 can be retracted back along catheter 528 to expose deployment system 522 (as illustrated in FIG. 5C). Retraction of catheter sleeve 564 can expose catch feature 542 of the distal end of floss wire 526.

As illustrated, deployment system 522 is delivered with an orientation on catheter 528 such that first branching portion 508 and second branching portion 510 of expandable branching stent prosthesis 500 are oriented corresponding to first branch 518 and second branch 520 of branching implant site 512.

It is also noted that floss wire 526 extends back from deployment system 522 and to the insertion site corresponding to the incision(s) for access to first branch 518.

With further similarity to FIG. 1C, in alternative cases, it may be that deployment system 522 is not covered by catheter sleeve 564 during placement. In such a case, catheter 528 on which deployment system 522 is mounted (e.g., via crimping) would be manipulated to extend outward from catheter delivery system 560 until catheter 528 is located at the desired location.

As was discussed with respect to FIGS. 3A-3B, in some embodiments, first branching portion 508 can extend further proximally than second branching portion 510. Accordingly, in some embodiments, catheter sleeve 564 can be retracted such that catch feature 542 is exposed, and first branching portion 508 remains within catheter sleeve 564, which is described in more detail in regard to FIG. 5D.

FIG. 5D illustrates an optional deployment stage for deployment system 522’ of according to embodiments herein. In the illustrated embodiment, deployment system 522’ can be seen as it has reached into trunk 516 of branching implant site 512, and catheter sleeve 564’ has been retracted a distance along catheter 528’.

As was discussed with respect to FIGS. 1C-5C, once deployment system 522’ has reached the appropriate distance into the trunk, catheter sleeve 564’ of deployment system 522’ can be retracted back along catheter 528’ to expose deployment system 522’ (as illustrated in FIG. 5C). Retraction of catheter sleeve 564’ can expose catch feature 542’ of the distal end of floss wire 526’.

In some embodiments, as discussed, first branching portion 508’ can extend proximally further than second branching portion 510’. In some cases, this can allow for—retraction of the catheter 528’, exposure of catch feature 542’, separation of the first branching portion 508’ and second branching portion 510’, and so on—without removing first branching portion 508’ from within the catheter 528’. This can allow for protection and facilitate resheathing of first branching portion 508’.

After release and separation of second branching portion 510’, in some embodiments, catheter 528’ can be advanced, or re-advanced to cover first branching portion 508’.

With further similarity to FIG. 1C, in alternative cases, it may be that deployment system 522 is not covered by catheter sleeve 564 during placement. In such a case, catheter 528 on which deployment system 522 is mounted (e.g., via crimping) would be manipulated to extend outward from catheter delivery system 560 until catheter 528 is located at the desired location.

FIG. 5E illustrates a snaring deployment stage for deployment system 522 of FIG. 5A, according to embodiments described herein. In the illustrated embodiment, deployment system 522 can be seen as it has reached into trunk 516 of branching implant site 512, and catheter sleeve 564 has been retracted a distance along catheter 528.

As discussed with respect to previous figures, once deployment system 522 has reached the appropriate distance into the trunk 516, a snare wire 568 can be introduced along second branch 520 to capture catch feature 542. In some cases, snare wire 568 can also be used to disengage second branching portion 510 from securement by first branching portion 508.

Once deployment system 522 is in the desired position, one or more incisions (not illustrated) giving access to second branch 520 of branching implant site 512 is made in the body. Then, a distal end of snare wire 568 is delivered to branching implant site 512 via second branch 520 e.g., using second catheter delivery system 570 through the access incision(s) for second branch 520. Snare wire 568 may extend back through second branch 520 of branching implant site 512 for to/through an insertion site corresponding to the incision(s) for access to second branch 520.

As illustrated, the distal end of snare wire 568 includes a snare feature 569 that is configured to interact with catch feature 542 of floss wire 526 in order to connect and/or snare the distal end of floss wire 526 together with the distal end of snare wire 568 at branching implant site 512. A practitioner manipulating floss wire 526 and/or snare wire 568 from their respective corresponding insertion sites may cause this connecting and/or snaring together to occur.

Once floss wire 526 and snare wire 568 have been connected, a practitioner manipulating floss wire 526 and/or snare wire 568 can disengage or disjoin second branching portion 510 from first branching portion 508. In some cases, first branching portion 508 may secure second branching portion 510 (as was discussed with respect to FIGS. 2-3B). To disengage, or dislodge, second branching portion 510, floss wire 526 can be fixed in place, and a force directed proximally with respect to snare wire 568 can be applied. Otherwise stated, the snare wire can be retracted, or pulled, back through the second branch. Such a force can cause second branching portion 510 to disengage, or dislodge, from first branching portion 508 (as seen in FIG. 5E).

After floss wire 526 and snare wire 568 are connected together (as illustrated in FIG. 5E), snare wire 568 may be pulled back along second branch 520 in order to bring the distal end of floss wire 526 through second branch 520 and out through the insertion site/incision for the access to second branch 520, as has been described.

FIG. 5F illustrates a contralateral, sheath-docking stage for deployment system 522 of FIG. 5A, according to embodiments described herein. In the illustrated embodiment, a catheter sleeve 572 of second catheter delivery system 570 can be advanced over second branching portion 510 to reach a bifurcation point 509 of stent prosthesis 500.

In some embodiments, catheter sleeve 572 can be advanced over the floss wire 526 and over second branching portion 510. For example, in embodiments, catheter sleeve 572 can be advanced up second branch 520 and over second branching portion 510. For example, in use, doctors may feed an introducer sheath up second branch 520 and over second branching portion 510 to make pathway for second branching portion 510 rather than extending second branching portion 510 down into second branch 520 with no protection, as second branching portion 510 may catch on calcification within second branch 520.

Covering second branching portion 510 via catheter sleeve 572 can serve to reduce damage or abrasions to second branching portion 510 as stent prosthesis 500 is seated within branching implant site 512. Otherwise stated, catheter sleeve 572 can protect second branching portion 510 from damage or abrasions as it is transitioned from within trunk 516, to a seating location within second branch 520.

In some embodiments, a protective shroud 530 (as was discussed with respect to FIGS. 1C-4B) can be configured to protect a distal end of second branching portion 510 as catheter sleeve 572 is advanced over second branching portion 510.

FIG. 5G illustrates an ipsilateral, sheath-docking stage for deployment system 522 of FIG. 5A, according to embodiments described herein. As was discussed with respect to FIG. 5F, in a similar fashion, and in some embodiments, catheter sleeve 564 of catheter delivery system 560 can be advanced, or re-advanced, over first branching portion 508 to reach bifurcation point 509 of stent prosthesis 500. Thus, in some embodiments, both first branching portion 508 and second branching portion 510 can be sheathed, prior to seating of stent prosthesis 500.

As discussed, in some cases, catheter sleeve 564 is retracted such that first branching portion 508 is wholly distal to catheter sleeve 564. In such cases, catheter sleeve 564 can be advanced over the proximal end of first branching portion 508. In some embodiments, a catheter sleeve 564 can be advanced over floss wire 526 over first branching portion 508. For example, in embodiments, catheter sleeve 564 can be advanced up first branch 518 and over first branching portion 508. For example, in use, doctors may advance an introducer sheath up first branch 518 and over first branching portion 508 to make pathway for first branching portion 508 rather than extending first branching portion 508 down into first branch 518 with no protection, as first branching portion 508 may catch on calcification within first branch 518.

As previously discussed with respect to FIGS. 2-4B, in some cases, distal end 538 of first branching portion 508 can be protected via a protective portion of the balloon within deployment system 522, as catheter sleeve 564 is advanced. In alternate embodiments, a protective shroud (such as protective shroud 130 described with respect to FIG. 1C and second branching portion 110) can be used.

Alternatively, as discussed, in some cases first branching portion 508 may not be entirely removed from within catheter sleeve 564 (e.g., such as when first branching portion 508 is of a longer or larger size, as discussed with respect to FIGS. 3A-B).

Covering first branching portion 508 via catheter sleeve 564 can serve to reduce damage or abrasions to first branching portion 508 as stent prosthesis 500 is seated within branching implant site 512. Otherwise stated, catheter sleeve 564 can protect first branching portion 508 from damage or abrasions as it is transitioned from within trunk 516, to the seating location within first branch 518. Thus, in some embodiments, both first branching portion 508 and second branching portion 510 can be sheathed, prior to seating of expandable branching stent prosthesis 500 into the branching implant site 512.

FIG. 5H illustrates a seating stage for deployment system 522 of FIG. 5A, according to embodiments described herein. Once floss wire 526 (and/or the and the snare wire 568) are adjusted and/or first branching portion 508 and/or second branching portion 510 are sheathed, floss wire 526 can pulled, and/or manipulated, etc. (e.g., by the practitioner using ends of floss wire 526 and/or the snare wire at the respective insertion site for each) to seat first branching portion 508 of expandable branching stent prosthesis 500 along first branch 518 of branching implant site 512 and second branching portion 510 of expandable branching stent prosthesis 500 along second branch 520 of branching implant site 512.

The pulling of floss wire 526 through second branch 520 may cause first branching portion 508 of expandable branching stent prosthesis 500 to become seated along first branch 518 of branching implant site 512 and second branching portion 510 of expandable branching stent prosthesis 500 to become seated along second branch 520 of branching implant site 512. Note that if necessary, after the distal end of second branch 520 is exposed to the practitioner through the insertion site/incision for the access to second branch 520, further manipulation of expandable branching stent prosthesis 500 may be performed using both ends of floss wire 526 at the respective insertion site for each end in order to achieve the appropriate seating of first branching portion 508 and second branching portion 510 of expandable branching stent prosthesis 500 in this manner. As provided above, a sheath may be advanced up second branch 520 and/or over second branching portion 510 to clear a pathway for second branching portion 510 as second branching portion 510 is pulled down from trunk 516 into second branch 520.

In some embodiments, both ends of floss wire 526 may be pulled simultaneously which displaces floss wire 526 to seat expandable branching stent prosthesis 500 in branching implant site 512. In some embodiments, one end of floss wire 526 may be fixed or locked so that only opposite end of floss wire 526 may be pulled to seat expandable branching stent prosthesis 500 in branching implant site 512. In other words, one end of floss wire 546 may be locked at a hub (not shown).

Once seated, if first branching portion 508 and/or second branching portion 510 have been sheathed via catheter sleeves (as discussed within FIGS. 5F-5G), the sleeves may be retracted or removed or withdrawn; expandable branching stent prosthesis 500 can thus be disposed in the manner illustrated in FIG. 5H. With deployment system 522 in the illustrated position, it may be that the expandable branching stent prosthesis 500 of deployment system 522 can be usefully deployed within branching implant site 512.

FIG. 5I illustrates a partially expanded stage for deployment system 522 of FIG. 5A, according to embodiments described herein. Within the illustrated embodiment, balloon 524 is inflated (e.g., the practitioner may manipulate catheter delivery system 560 for balloon 524 to cause inflation of balloon 524). Inflation of balloon 524 expands trunk portion 506 and first branching portion 508, such that they deploy against walls of trunk 516 and first branch 518. Balloon 524 may then be deflated. While balloon 524 is inflated, blood flow through branching implant site 512 may be shortly inhibited. Trunk portion 506, first branching portion 508, and second branching portion 510 are each individually and independently expandable.

In some embodiments, balloon 524 may be attached to catheter 528, and may be inflatable and/or deflatable at the option of a practitioner operating the catheter delivery system 560 via a connection between balloon 524 and catheter 528 that can communicate fluid between catheter 528 and balloon 524.

FIG. 5J illustrates a partially expanded stage for deployment system 522 of FIG. 5A, according to embodiments described herein. Within the illustrated embodiment, balloon 524 (as was seen in FIG. 5I), can be deflated and/or removed.

In some cases, fluid (e.g., blood) can begin to flow through trunk portion 506 and first branching portion 508 after the balloon has been deflated. In some embodiments, although second branching portion 510 may not yet be inflated, lumen 544 of second branching portion 510 can serve to allow fluid flow through second branching portion 510. Otherwise stated, expanding trunk portion 506 and/or first branching portion 508 can open a proximal end of lumen 544 that was previously closed.

With expandable branching stent prosthesis 500 partially deployed and balloon 524 serving no further use, catheter 528 may be retracted out from branching implant site 512 and/or out of the body altogether (while the partially deployed expandable branching stent prosthesis 500 is left in place).

FIG. 5K illustrates a fully expanded stage for deployment system 522 of FIG. 5A, according to embodiments described herein. As illustrated in FIG. 5A, balloon delivery device 580 is delivered through the insertion site corresponding to second branch 520 and along floss wire 526 up second branch 520 of branching implant site 512 preparatory to insertion into second branching portion 510 of expandable branching stent prosthesis 500.

Balloon delivery device 580 includes a balloon 582 attached to balloon delivery device 580 and that is inflatable and/or deflatable via balloon delivery device 580 (e.g., via a practitioner's operation of balloon delivery device 580).

Note that due to lumen 544 of second branching portion 510 of expandable branching stent prosthesis 500, it is feasible to deliver balloon 582 into second branching portion 510 of expandable branching stent prosthesis 500 by coming up second branch 520 of branching implant site 512 along floss wire 526. Note that in some embodiments, should lumen 544 be absent, a dilator or dilation device may be used prior to introduction of balloon 582.

Balloon delivery device 580 may be positioned (e.g., by the practitioner manipulating balloon delivery device 580 from insertion site for second branch 520) consistent with the expansion/deployment of second branching portion 510 of expandable branching stent prosthesis 500 by balloon 582 of balloon delivery device 580.

Balloon delivery device 580 may be positioned (e.g., by the practitioner manipulating balloon delivery device 580 from the insertion site for second branch 520) consistent with the expansion/deployment of second branching portion 510 of expandable branching stent prosthesis 500 by balloon 582 of balloon delivery device 580.

Once balloon delivery device 580 is positioned, balloon delivery device 580 may be operated to inflate balloon 582 (e.g., using a control of balloon delivery device 580). Inflation of balloon 582 expands second branching portion 510 of expandable branching stent prosthesis 500, such that second branching portion 510 deploys against walls of second branch 520 of branching implant site 512. Balloon 582 may then be deflated, and balloon delivery device 580 removed away from branching implant site 512 and/or the body altogether.

FIG. 5L illustrates a post-deployment stage for deployment system 522 of FIG. 5A, according to embodiments described herein. In the illustrated embodiment, additional amounts of floss wire 526 are introduced to form loop 586. Loop 586 can aid in refining the positioning and seating of stent prosthesis 500, after initial deployment or expansion.

As may be seen, in the present embodiment, floss wire 526 is not necessarily pulled back along second branch 520 in order to bring the distal end of floss wire 526 through second branch 520 and out through the insertion site/incision for access to second branch 520.

In some cases, additional lengths of floss wire can be introduced from first branch 518 and/or second branch 520. These additional lengths can form slack, or loop 586.

In some cases, after loop 586 has been formed, post-deployment, or follow-up expansion methods can be introduced. For example, in embodiments, a third, fourth, or any number of additional balloons can be introduced into first branch 518 and/or second branch 520, and/or trunk 516. In some cases, additional expansion methods can serve in properly positioning or expanding the stent prosthesis.

In some cases, loop 586 can aid by providing substantially straightened portions 590 and/or 592 that can facilitate balloon placement. For example, in embodiments, straightened portions 590 and/or 592 can allow enhanced uniformity and penetration via any follow-up balloons, with respect to first branch 518 and/or second branch 520.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A method of manipulating a deployment system, the method comprising:
delivering to a branching implant site comprising a trunk, a first branch, and a second branch a deployment system via the first branch, the deployment system comprising:
an expandable stent prosthesis comprising:
a trunk portion;
a first branching portion proximally attached to the trunk portion; and
a second branching portion proximally attached to the trunk portion;
a floss wire comprising:
a proximal portion disposed in the first branching portion;
a bend disposed at a bifurcation point of the stent prosthesis; and
a distal portion disposed in the second branching portion;
displacing the floss wire to seat the expandable stent prosthesis within the branching implant site such that the first branching portion is disposed within the first branch, the trunk portion is disposed at the trunk, and the second branching portion is disposed in the second branch; and
expanding the expandable stent prosthesis within the branching implant site.

2. The method of claim 1, further comprising introducing an excess length of the floss wire into the branching implant site such that the bend is displaced distally from the bifurcation point into the trunk portion.

3. The method of claim 2, wherein introducing the excess length of the floss wire into the branching implant site comprises introducing excess length of the floss wire into the proximal portion of the floss wire.

4. The method of claim 2, wherein introducing the excess length of the floss wire into the proximal portion increases a longitudinal distance of the proximal portion of the floss wire.

5. The method of claim 2, wherein introducing the excess length of the floss wire into the branching implant site comprises introducing excess length of the floss wire into the distal portion of the floss wire.

6. The method of claim 2, wherein introducing the excess length of the floss wire into the distal portion increases a longitudinal distance of the distal portion of the floss wire.

7. The method of claim 1, further comprising:
introducing a first balloon into the second branching portion via the distal portion of the floss wire; and
inflating the first balloon.

8. The method of claim 7, wherein a distal end of the first balloon is concentric with an outer diameter of the distal portion of the floss wire.

9. The method of claim 7, wherein the first balloon has a linear profile when the first balloon is inflated.

10. The method of claim 7, wherein the first balloon comprises a longitudinal axis that coincides with a longitudinal axis of the distal portion of the floss wire.

11. The method of claim 7, wherein a portion of the first balloon extends into the trunk when the first balloon is inflated.

12. The method of claim 1, further comprising:
introducing a second balloon into the first branching portion via the proximal portion of the floss wire; and
inflating the second balloon.

13. The method of claim 12, wherein a distal end of the second balloon is concentric with an outer diameter of the proximal portion of the floss wire.

14. The method of claim 12, wherein the second balloon has a linear profile when the second balloon is inflated.

15. The method of claim 12, wherein a portion of the second balloon extends into the trunk portion when the second balloon is inflated.

16. The method of claim 12, wherein the second balloon comprises a longitudinal axis that coincides with a longitudinal axis of the proximal portion of the floss wire.

17. The method of claim 1, further comprising pulling simultaneously on both ends of the floss wire during displacement to seat the branching stent prosthesis.

18. The method of claim 1, further comprising locking one end of the floss wire and pulling on the other end during displacement to seat the branching stent prosthesis.
